(19) United States
(12) Patent Application Publication
(10) Pub. No.: US 2016/0091921 A1
(43) Pub. Date: Mar. 31, 2016

(54) SMART WATCH WITH UV DETECTOR

(71) Applicant: Cheng Uei Precision Industry Co., Ltd., New Taipei City (TW)

(72) Inventor: CHENG LEE, New Taipei City (TW)

(21) Appl. No.: 14/548,263

(22) Filed: Nov. 19, 2014

(30) Foreign Application Priority Data
Sep. 30, 2014 (TW) 103217388

(51) Int. Cl. G06F 1/16; A61B 5/024; G01J 1/42

(52) U.S. Cl. CPC G06F 1/163; G01J 1/429; A61B 5/02438; A61B 5/02444

(57) ABSTRACT

A smart watch with an ultraviolet (UV) detector includes a base body, a display module, a UV detector module and a processor module. The base body includes a top housing and a watchband. The top housing defines a receiving chamber. At least one lateral face of the top housing is an inclined face. The inclined face is recessed inward and slantwise downward to form a groove. The display module is disposed in the receiving chamber. The UV detector module is disposed in the groove and exposed outward through the groove, wherein in use, the UV detector module is located outward and upward back to a user's body for gaining UV information. The processor module is disposed in the receiving chamber and electrically connected to the display module and the UV detector module. The processor module processes the UV information and then displays processed results on the display module.

### CROSS-REFERENCE TO RELATED APPLICATION

The present application is based on, and claims foreign priority from, Taiwan Patent Application No. 103217388, filed Sep. 30, 2014, the disclosure of which is hereby incorporated by reference herein in its entirety.

### BACKGROUND OF THE INVENTION

1. Field of the Invention

The present invention relates to a watch, and more particularly to a smart watch with an ultraviolet (UV) detector.

2. The Related Art

Currently, watches are items needed in daily life, and their function become more and more complex due to a great advance in technology. For example, user can use a smart watch to do UV measurement in outdoor sports, so as to avoid skin damage caused by excessive exposure outdoors. The smart watch has a flat screen and a UV sensor. The UV sensor is mounted on the display screen, and an exposed face of the UV sensor is flush with the screen.

However, when the user looks at the screen of the smart watch, the user will turn the wrist inward to make the eyes perpendicular to the screen. But this action makes the exposed face of the UV sensor slantwise downward. As a result, it affects the UV light reception and causes an inaccurate UV measurement result. Furthermore, because the UV sensor is mounted on the screen, the exposed face of the UV sensor is easily obscured when the user touches the screen. So that further affects the UV light reception and further causes an inaccurate UV measurement result.

### SUMMARY OF THE INVENTION

Accordingly, an object of the present invention is to provide a smart watch with an ultraviolet (UV) detector. The smart watch with UV detector includes a base body, a display module, a UV detector module and a processor module. The base body includes a top housing and a watchband. The top housing is mounted to the watchband and protrudes on top of the watchband. The top housing defines a receiving chamber therein and a window. The window is opened through a top face of the top housing and communicated with the receiving chamber. At least one lateral face of the top housing is an inclined face connected with the watchband along length direction of the watchband. The inclined face is recessed inward and slantwise downward to form a groove communicating with the receiving chamber. The display module is disposed in the receiving chamber of the top housing. A top of the display module is fitted in the window of the top housing. The UV detector module is disposed in the groove of the top housing and exposed outward through the groove, wherein in use, the UV detector module is located outward and upward back to a user's body for gaining UV information. The processor module is disposed in the receiving chamber of the top housing. The processor module is electrically connected to the display module and the UV detector module. The processor module processes the UV information from the UV detector module and then displays processed results of the UV information on the display module.

As described above, the UV detector module is disposed in the groove of the top housing, and is generally located at outside of the wrist of the user in use. Thus, when sight of the user is perpendicular to the display module for needing about UV index, an exposed face of the UV detector module is just outward and upward to receive the UV information, which can effectively improve UV light reception. Furthermore, when some actions are done on the display module, it will not block UV light path, so as not to affect displayed results of the UV index on the display module.

### BRIEF DESCRIPTION OF THE DRAWINGS

The present invention will be apparent to those skilled in the art by reading the following description thereof, with reference to the attached drawings, in which:

FIG. 1 is a perspective view showing a smart watch with a UV detector according to an embodiment of the present invention;

FIG. 2 is an exploded view of the smart watch with the UV detector shown in FIG. 1;

FIG. 3 is a sectional view of the smart watch with the UV detector in FIG. 1;

FIG. 4 is a partial enlarged view of the smart watch with the UV detector in FIG. 3; and

FIG. 5 is an electrical diagram of the smart watch with the UV detector shown in FIG. 1.

### DETAILED DESCRIPTION OF THE EMBODIMENT

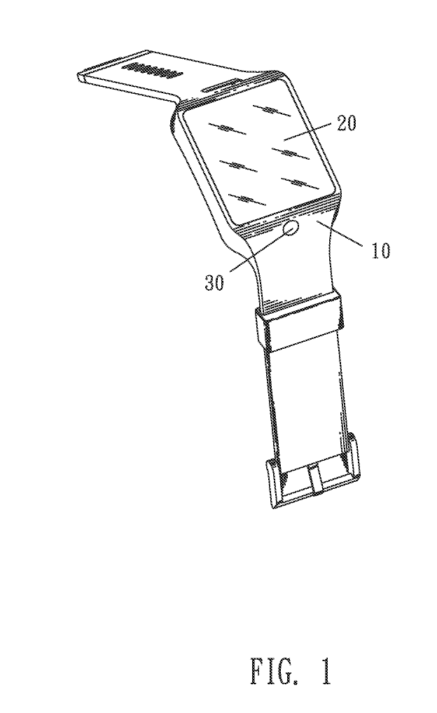

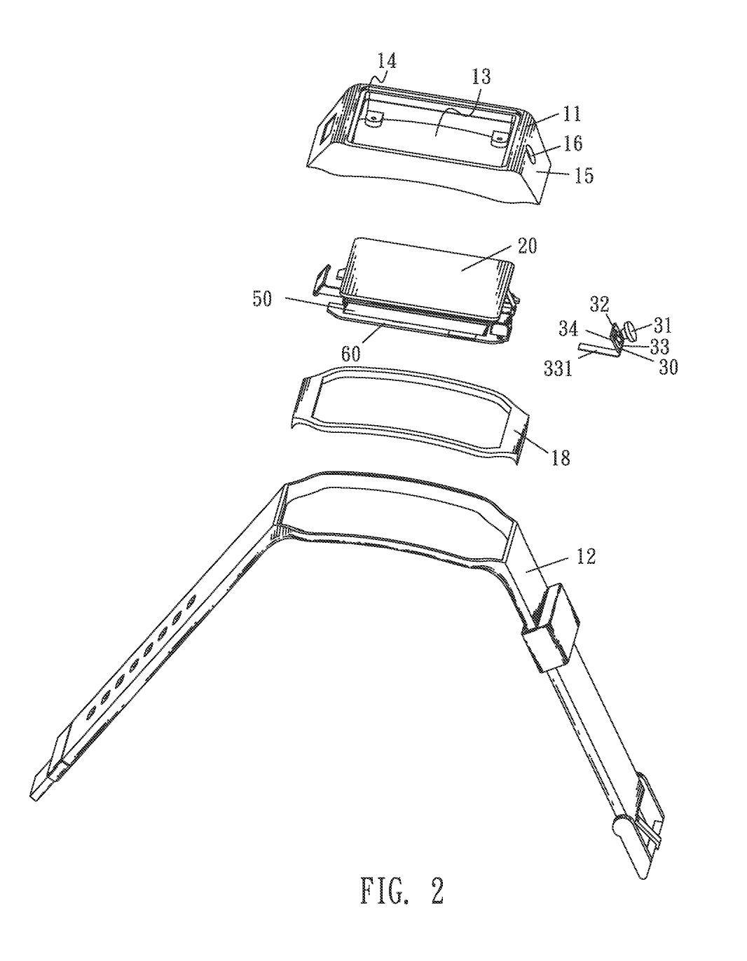

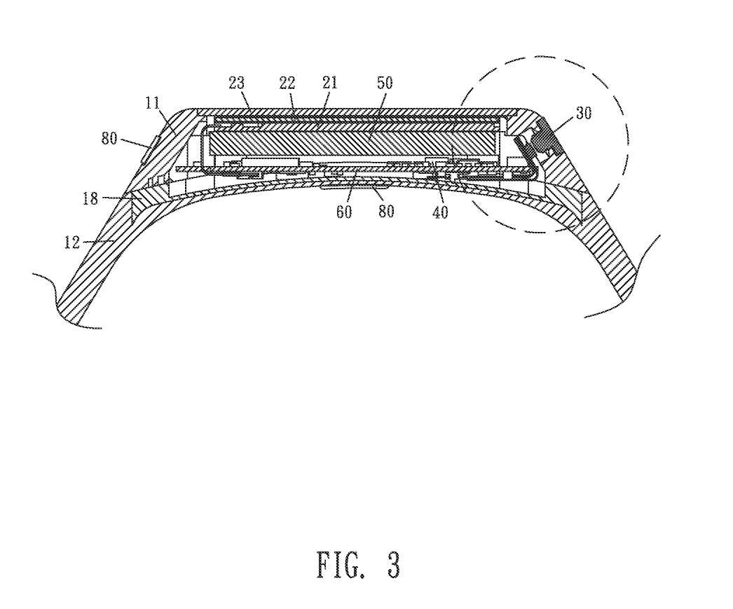

Referring to FIG. 1, FIG. 2 and FIG. 3, the smart watch with an ultraviolet (UV) detector 100 according to the present invention includes a base body 10, a display module 20, a UV detector module 30, a processor module 40, a battery 50 and a main circuit board 60.

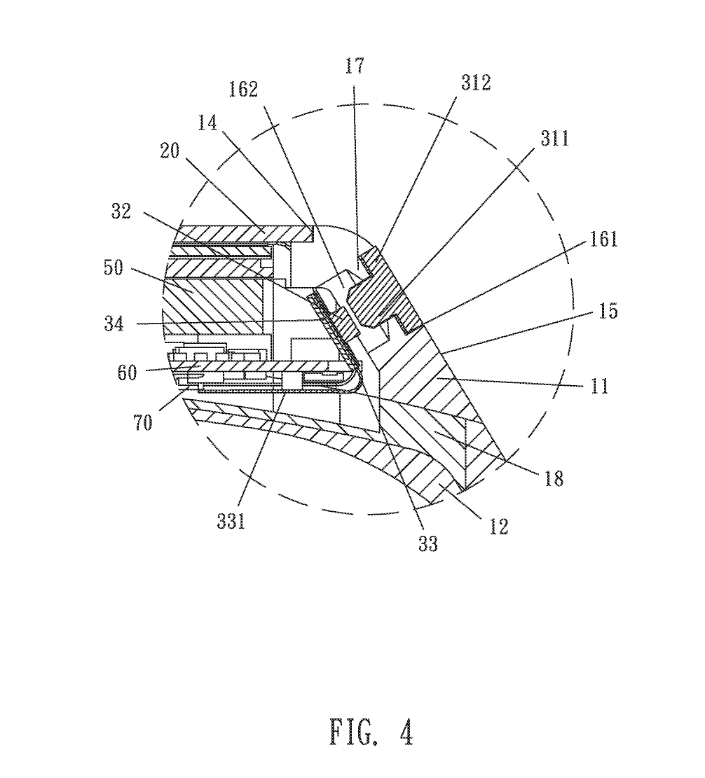

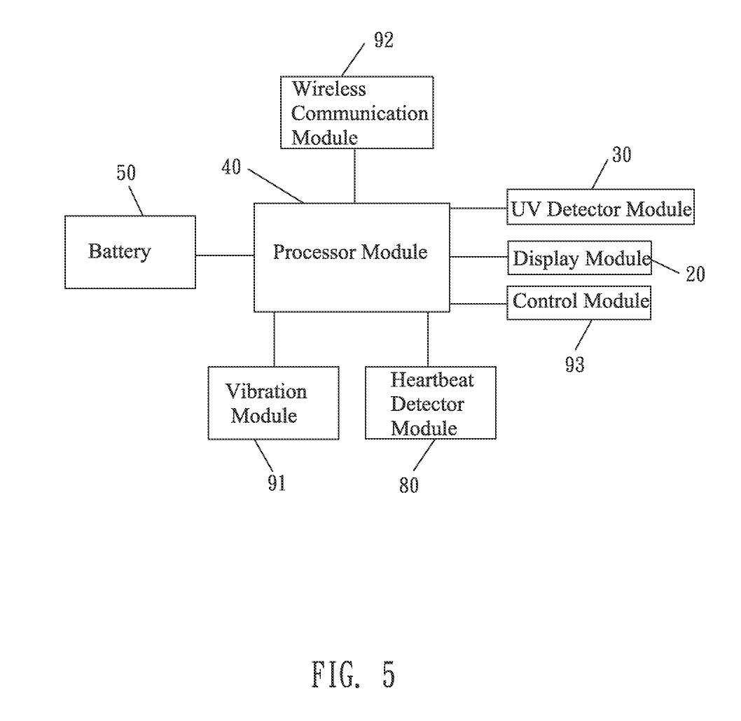

Referring to FIG. 1, FIG. 2, FIG. 3, FIG. 4 and FIG. 5, the base body 10 includes a top housing 11, a watchband 12 and a lower housing 18. The top housing 11 is mounted to the watchband 12 and protrudes on top of the watchband 12. The lower housing 18 is combined to a bottom of the top housing 11 and embedded in the watchband 12 for mounting the top housing 11 to the watchband 12. The top housing 11 defines a receiving chamber 13 therein and a window 14. The window 14 is opened through a top face of the top housing 11 and communicated with the receiving chamber 13. At least one lateral face of the top housing 11 is an inclined face 15 connected with the watchband 12 along length direction of the watchband 12. In this invention, the inclined face 15 is not limited to an inclined plane in the strict sense, and it can also be a curved structure and so on. The inclined face 15 is recessed inward and slantwise downward to form a groove 16 communicated with the receiving chamber 13. A substantial middle of inner sidewall of the groove 16 protrudes inward to form a ring-shaped propping ledge 17. The propping ledge 17 divides the groove 16 into an upper groove 161 and a lower groove 162.

The display module 20 is disposed in the receiving chamber 13 of the top housing 11. A top of the display module 20 is fitted in the window 14 of the top housing 11. Referring to FIG. 3, the display module 20 includes a display screen 21, a diffuser 22 and a cover glass 23 which are positioned in the receiving chamber 13 of the top housing 11 from the bottom up. The cover glass 23 is fitted in the window 14 of the top housing 11 to be exposed outward.

The UV detector module 30 is disposed in the groove 16 of the top housing 11 and exposed outward through the groove 16. Preferably, the UV detector module 30 is exposed flush with the inclined face 15 of the top housing 11, wherein in use, the UV detector module 30 is located outward and upward back to a user's body for gaining UV information. Referring to FIG. 2 and FIG. 4, the UV detector module 30 includes a UV detector module circuit board 33, a UV detector 32 and a UV detector lens 31 which are positioned in the groove 16 of the top housing 11 from the inside out. The UV detector lens 31 is exposed outward through the groove 16 and is flush with the inclined face 15 of the top housing 11. The UV detector 32 is electrically located on the UV detector module circuit board 33 and faces to the UV detector lens 31. The UV detector lens 31 has a cylindrical body portion 311. The periphery of an upper end of the body portion 311 extends outward to form an umbrella portion 312. The body portion 311 stretches in the lower groove 162, the umbrella portion 312 is positioned in the upper groove 161 and an inside face of the umbrella portion 312 is fixed on the propping ledge 17 by adhesive.

The UV detector module 30 further includes a stiffener 34. The UV detector module circuit board 33 is a flexible printed circuit board of which an inner side is fixedly bonded to the stiffener 34, thereby strengthening the hardness of the UV detector module circuit board 33, thus reducing the problem of surface mount and assembling. A lower end of the UV detector module circuit board 33 is bent inward to form an elastic section 331. The elastic section 331 is electrically connected to a connector 70 which is connected to the main circuit board 60 to realize electrical connections between the UV detector module 30 and the main circuit board 60.

The processor module 40 is disposed in the receiving chamber 13 of the top housing 11 and electrically connected to the display module 20 and the UV detector module 30. The processor module 40 processes the UV information from the UV detector module 30 and then displays processed results of the UV information on the display module 20.

The battery 50 and the main circuit board 60 are disposed in the receiving chamber 13 of the top housing 11. The battery 50 is located on the main circuit board 60 and electrically connected to the main circuit board 60. The display module 20 is located on the battery 50 and electrically connected to the battery 50, and the battery 50 provides power for the display module 20 and other components of the smart watch with UV detector 100.

Referring to FIG. 5, the smart watch with UV detector 100 further includes a heartbeat detector module 80, a vibration module 91, a wireless communication module 92 and a control module 93. The heartbeat detector module 80 is disposed in the base body 10. The heartbeat detector module 80 is used to detect some physiological signals, such as user's heartbeat, heart rate variability analysis and emotional index, and then transmit the physiological signals to the processor module 40. The processor module 40 controls the vibration module 91 to generate vibrations. The communication is built between the processor module 40 and an external electronic device through the wireless communication module 92 to transmit or receive data. The control module 93 includes a touch circuit (not shown). The touch circuit is in conjunction with the display module 20 to constitute a touch panel (not shown) so as to control the processor module 40 to realize operation and setting of the smart watch with UV detector 100.

Referring to FIG. 1, FIG. 2, FIG. 3 and FIG. 4, when the smart watch with UV detector 100 is in use, the UV detector 32 receives different wavelength ultraviolet, then the processor module 40 gains different UV information of the different wavelength ultraviolet and further processes the different UV information into UV index which will be displayed on the display module 20. The user takes different protection measures according to the UV index. As shown in the following table, 0˜2 stands for a low UV index, 3-5 stands for a moderate UV index, 6˜7 stands for a high UV index, 8˜10 stands for an extremely high UV index, and the index of 11 is the limit. The UV detector module 30 is generally located at outside of the wrist of the user when it is in use. When the user looks at the UV index, the user will turn the wrist inward to make sight of the eyes perpendicular to the display module 20. At this time, an exposed face of the UV detector module 30 is just outward and upward to receive the different UV information, which can effectively improve UV light reception. Furthermore, when some actions are done on the display module 20, it will not block UV light path, so as not to affect the displayed results of the UV index on the display module 20.

| UV Index | UV wavelength |
|---|---|
| 0 | ≦747 |
| 1 | 748~1494 |
| 2 | 1495~2241 |
| 3 | 2242~2988 |
| 4 | 2989~3735 |
| 5 | 3736~4482 |
| 6 | 4483~5229 |
| 7 | 5230~5976 |
| 8 | 5977~6723 |
| 9 | 6724~7470 |
| 10 | 7471~8217 |
| 11 | ≧8218 |

As described above, the UV detector module 30 is disposed in the groove 16 of the top housing 11, and is generally located at outside of the wrist of the user in use. Thus, when sight of the user is perpendicular to the display module 20 for needing about the UV index, the exposed face of the UV detector module 30 is just outward and upward to receive the UV information, which can effectively improve UV light reception. Furthermore, when some actions are done on the display module 20, it will not block UV light path, so as not to affect the displayed results of the UV index on the display module 20.

1. A smart watch with an ultraviolet (UV) detector, comprising:
a base body including a top housing and a watchband, the top housing being mounted to the watchband and protruding on top of the watchband, the top housing defining a receiving chamber therein and a window, the window being opened through a top face of the top housing and communicated with the receiving chamber, at least one lateral face of the top housing being an inclined face connected with the watchband along length direction of the watchband, the inclined face being recessed inward and slantwise downward to form a groove communicating with the receiving chamber;
a display module disposed in the receiving chamber of the top housing, a top of the display module being fitted in the window of the top housing;
a UV detector module disposed in the groove of the top housing and exposed outward through the groove, wherein in use, the UV detector module is located outward and upward back to a user's body for gaining UV information; and
a processor module disposed in the receiving chamber of the top housing, the processor module being electrically connected to the display module and the UV detector module, the processor module processing the UV information from the UV detector module and then displaying processed results of the UV information on the display module.

2. The smart watch with the UV detector as claimed in claim 1, wherein the UV detector module is exposed flush with the inclined face of the top housing.

3. The smart watch with the UV detector as claimed in claim 1, wherein the UV detector module includes a UV detector module circuit board, a UV detector and a UV detector lens which are positioned from inside to outside in the groove of the top housing, the UV detector lens is exposed through the groove, the UV detector is electrically located on the UV detector module circuit board and faces to the UV detector lens.

4. The smart watch with the UV detector as claimed in claim 3, wherein a substantial middle of inner sidewall of the groove protrudes inward to form a ring-shaped propping ledge, the propping ledge divides the groove into an upper groove and a lower groove, the UV detector lens has a cylindrical body portion, the periphery of an upper end of the body portion extends outward to form an umbrella portion, the body portion stretches in the lower groove, the umbrella portion is positioned in the upper groove and an inside face of the umbrella portion is fixed on the propping ledge by adhesive.

5. The smart watch with the UV detector as claimed in claim 3, wherein the UV detector module further includes a stiffener, the UV detector module circuit board is a flexible printed circuit board of which an inner side is fixedly bonded to the stiffener.

6. The smart watch with the UV detector as claimed in claim 5, wherein the smart watch with UV detector further includes a main circuit board and a connector which is connected to the main circuit board, a lower end of the UV detector module circuit board is bent inward to form an elastic section, the elastic section is electrically connected to the connector.

7. The smart watch with the UV detector as claimed in claim 3, wherein the periphery of the UV detector module circuit board is fixed on sidewall of the groove by adhesive.

8. The smart watch with the UV detector as claimed in claim 1, wherein the smart watch with UV detector further includes a heartbeat detector module, the heartbeat detector module is disposed in the base body, the heartbeat detector module is used to detect some physiological signals, such as user's heartbeat, heart rate variability analysis and emotional index, and then transmit the physiological signals to the processor module.

9. The smart watch with the UV detector as claimed in claim 1, wherein the smart watch with UV detector further includes a lower housing combined to a bottom of the top housing and embedded in the watchband for mounting the top housing to the watchband.
